(19) United States
(12) Patent Application Publication
(10) Pub. No.: US 2007/0231375 A1
(43) Pub. Date: Oct. 4, 2007

(54) Liposome combination and the use thereof

(75) Inventors: Tsuimin Tsai, Taipei City (TW); Chin-Tin Chen, Taipei City (TW); Ruey-Long Hong, Taipei City (TW); Hong-Da Wu, Taipei City (TW)

Correspondence Address: BIRCH STEWART KOLASCH & BIRCH, PO BOX 747, FALLS CHURCH, VA, 22040-0747, US

(73) Assignee: Taipei Medical University

(21) Appl. No.: 11/589,198

(22) Filed: Oct. 30, 2006

(30) Foreign Application Priority Data
Apr. 3, 2006 (TW) 095111731

(51) Int. Cl. A61K 9/127; A61K 31/555; A61K 31/409; A61N 5/06

(52) U.S. Cl. 424/450; 514/185; 514/410; 607/86

(57) ABSTRACT

The present invention relates to a liposome combination, which wrapped hydrophilic drugs in water layer and wrapped hydrophobic drugs in lipid bilayer; hydrophobic drugs are photosensitizers. Using light with appropriate wavelength to activate the photosensitizer in the hydrophobic layer can result in the production of singlet oxygen and the free radical, and cause the oxidizing and breaking of the carbon chain of the phospholipid, and influences the stability of the liposome and the releases of the drug. The singlet oxygen and the free radical will attack the cancer cells at the same time as a result of combining the photodynamic- and chemo-effects.

### BACKGROUND OF THE INVENTION

1. Field of the Invention

This invention relates to a new liposome releasing control system, and more specifically which contains the photosensitive substance encapsulated in the lipid bilayer of liposome, and simultaneously encapsulated the release drug in its hydrophilic layer. The action mechanism of this invention depends on light-induced photochemical reaction to subsequently enhance the drug release of said liposome and exert its cytotoxic effect to cancer cells. This invention further relates a new cancer drug formulation and its therapy thereof.

2. Description of the Related Art

The present invention is a new liposome releasing control system and cancer therapy thereof.

Bangham et al, (1965) discovered the lipid-based small permeable ball; Sessa and Weissmann (1968) name the small ball as “liposome” and make a definition: the liposome is a small vesicle comprising single to multiple lipid bilayer.

Science 1970, it is presumed that the liposome is a good drug carrier with the following characteristics:

(1) Liposome is made from phospholipids. Its composition is cell membrane-like and can decompose in vivo, therefore, it is non-toxicity, and can be used repeatedly;

(2) The drug encapsulated in the liposome that can be released at slowly rate. Liposomal encapsulation of the drug can enhance the therapeutic effect by changing the pharmacokinetic profile of the drug;

(3) Reduced side-effect may be achieved when the drug is encapsulated in the liposome;

(4) The liposomes can suit many situations by methods including varying the lipid composition, particle size, structure, preparation method of the liposomes.

In 1990's, it was discovered that using PEG-PE connected to the surface of the disteroylphosphotidyl-choline liposomes, and reduced the particle diameter of the liposomes to 200 nm or less, such liposomes can circulate a long time in the blood system. These long-circulating liposomes can escape the clearance of endothelial cell and have higher accumulation in tumor sites (10 times higher than normal tissue). The Taiwan Patent Appl. No. 088101359 shows that adding more PEG-PE in liposome formula can enhance liposome's storage time.

The long-circulating liposomes have been commercialized and the products have sold the market already, for example, Lipo-Dox® and Doxil®. Lipo-Dox® and Doxil® was encapsulated doxorubicin both and undergo FDA authentication to permit using these formulation for cancer therapy at present. However, the defects of the present long-circulating liposome are as follows:

1. The liposomes were unable to release quickly after entering the target sites when the composition was saturate phospholipids with high quantity cholesterol, because the composition of the liposome is too steady.

2. The PEG-PE seems to hinder a drug releasing ability of the liposome.

3. The PEG-PE seems to hinder the ability of liposome to enter the tumor tissue and cell.

Many studies have shown that smaller liposomes can easily penetrate through the stratum corneum and the mucous membrane, and reach the target sites. Preventing the aggregation and fusion of the liposomes, and the unexpected leakage of the drug from the liposomes during the manufacturing and the storage condition are the important issues in the dosage form design. Therefore, a highly stable formulation that can precisely control the drug release is an important goal in the development of liposomes.

At present, many researchers have tried various ways to control the drug release of liposome, using the instability of the structure of liposomes.

Grossweiner (1980) used liposome made from Egg PC and DPPC to be the cell membrane model. Liposomes encapsulating a fluorescent element “eosin”, mixed with methyl blue solution, resulted in the release of eosin when exposed to light. The result shows using this method can release eosin effectively, and the free radical and super oxide produced from the excitation of methyl blue can attack the fatty acid chain of the phospholipids and resulted in unstable lipid bilayer. Here the photosensitizer is not inside the liposomes, thereof, the singlet oxygen and free radical must pass through the lipid bilayer to attack the fatty acid chain, and such traveling resulted in reduced photochemical effect.

The photodynamic therapy comprises three elements: photosensitizer, oxygen and light. The photosensitizer itself is almost non-toxic for human. When the photosensitizer accumulates at the tumor tissue, and exposed to light, then the photosensitizer will transform in to an excited state. The excited photosensitizer will transfer its energy to the surrounding oxygen and substances, and form the singlet oxygen and free radicals to kill the cells. There is one report shows that combining doxorubicin liposome and photodynamic treatment resulted in better tumor inhibition.

Although the current liposome manufacturing techniques are able to produce quite stable liposomes, there is still no satisfying ways to control the immediate release of the liposomes at the target sites. In addition, dual encapsulation of a photosensitizer and another water-soluble drug in the same liposome has not been invented yet. Therefore, the present invention provides a liposome with a photosensitizer and a chemical therapy drug. The liposome can release the drug rapidly by light activation, and has the ability to combine photodynamic- and chemo-therapies.

### SUMMARY OF THE INVENTION

The present invention advantageously fills the aforementioned need by providing a new release-controlled liposome system.

The purpose of the present invention is to provide a new release-controlled liposome system. More specifically, it depends on the photodynamic effect to enhance the drug release from the liposome and the elimination of cancer cells.

Another purpose of the present invention is to provide a new cancer drug form and its therapy thereof.

The present invention relates to an application system of liposome which encapsulated a photosensitizer and a hydrophilic substance. Said photosensitive liposome formulation is a stability of liposome and can controlled drug release from liposome, it also provides a co-carrier for photosensitizer and chemical therapeutic drug. After light activation, said liposome have not only the controlled release ability, but also the ability for photodynamic- and chemo-therapeutic effects.

The present invention is to design a photosensitive liposome; which uses higher stable phospholipids with saturated fatty acid and cholesterol, and encapsulates photosensitizer into the lipid bilayer. Said substance produced chemical reaction with phospholipids that via light activation to promote the drug release from the liposome. Due to the lipid bilayer structure of the liposome, the liposome of the present invention can encapsulates two substances: the hydrophilic drug encapsulated in the hydrophilic layer, and the photosensitizer encapsulated in the hydrophobic layer. Using light with appropriate wavelength to activate the photosensitizer in the hydrophobic layer can result in the production of singlet oxygen and the free radical, and cause the oxidizing and breaking of the carbon chain of the phospholipid, and influences the stability of the liposome and the releases of the drug. The singlet oxygen and the free radical will drift to the outside of liposome and attack the cancer cells at the same time as a result of combining the photodynamic- and chemo-effects.

The present invention relates to a liposome encapsulating photosensitizer in its lipid bilayer. The hydrophilic layer of said liposome encapsulates the model drug such as, but not limited to, Doxorubicin and Calcein, and the hydrophobic layer of said liposome encapsulates photosensitive substance such as, but not limited to, Hematoporphyrin (Hp) and Protoporphyrin (Pp).

The basic formulation of the present invention liposome combination is 1,2-Disteroyl-sn-Glycero-3-Phosphocholine (DSPC), Cholesterol, Polyethyleneglycol-derivated Distearoylphosphatidylethanolamine (PEG-DSPE), and encapsulates hydrophobic photosensitive substance such as, but not limited to, Hematoporphyrin (Hp) and Protoporphyrin (Pp) in its lipid bilayer, and encapsulated the hydrophilic drug in the hydrophilic layer. Said liposome combination can cause the fatty acid chain of phospholipids broken by singlet oxygen and free radical after light treatment. Said liposome combination can controlled the drug release, and enhance the cytotoxic effect of cancer cells after activated by light.

The present invention further relates to a method for using said liposome combination. The light source of the present invention is the light with long wave such as, but not limited to, 600 nm-670 nm. It activates the photosensitizer and influences the stability of liposome, then the liposome releases out the drug and the singlet oxygen and the free radical will drift to the outside of liposome and attack the cancer cells at the same time as a result of combining the photodynamic- and chemo-effects.

The present invention now will be described more fully hereinafter with reference to the accompanying drawings, which are intended to be read in conjunction with both this summary, the detailed description and any preferred and/or particular embodiments specifically discussed.

### BRIEF DESCRIPTION OF THE DRAWINGS

The present invention will become apparent upon reading of the following detailed description of the present invention in conjunction with the drawings, as follows:

FIG.1 is a diagram of the stability analysis of different liposomes stored at 4° C. according to the present invention; DSPC: Egg PC: Cholesterol: PEG-DSPE= [symbol] 8:2:1:0.2 μmole, (▪) 9:1:1:0.2 μmole, (▴) 10:0:1:0.2 μmole, (×) 10:0:2:0.2 μmole, (*) 10:0:3:0.2 μmole.

FIG. 2 is a diagram of drug release from the liposome in 37° C. PBS after light activating, the encapsulated drug is Hp.

FIG. 3 is a diagram of drug release from the liposome in 37° C. PBS after light activating, the encapsulated drug is PpIX.

FIG. 4 is a diagram of the lipid bilayer permeability change of the liposome in the absence of photosensitizer after 30 J/cm² light irradiation, the model drug used to monitor the permeability of the membrane is Doxorubicin.

FIG. 5 is a diagram of the lipid bilayer permeability change of the liposome in the absence of photosensitizer after 30 J/cm² light irradiation, the model drug used to monitor the permeability of the membrane is Calcein.

FIG. 6 is a diagram of the lipid bilayer permeability change of the liposome encapsulating a photosensitizer (Hp) after 30 J/cm² light irradiation, the model drug used to monitor the permeability of the membrane is Doxorubicin.

FIG. 7 is a diagram of the lipid bilayer permeability change of the liposome encapsulating a photosensitizer (Hp) after 30 J/cm² light irradiation, the model drug used to monitor the permeability of the membrane is Calcein;

FIG. 8 is a diagram of the toxicity of various formulation to CL1-0 cell Liposomal-Hp and free-Hp were added into the cell culture system and light irradiation was provided with doses from 0 J, 2 J, 4 J, 6 J, to 8 J;

FIG. 9 is a diagram of the toxicity of various formulation to A431 cells, Liposomal Doxorubicin (LD), Free Doxorubicin (FD), Liposomal-Dox-Hp (LDH),

Liposomal Hp (LH), and LDH treating with 1 J, 10 J, 20 J, and 30 J of light were compared;

FIG. 10 is a diagram of the toxicity of various formulation to CL1-0 cells.

Liposomal Doxorubicin (LD), Free Doxorubicin (FD), Liposomal-Dox-Hp (LDH), and LDH treated with 1 J, and 10 J of light were compared.

### DETAILED DESCRIPTION OF THE INVENTION

The present invention relates to a liposome encapsulates a photosensitive substance in its lipid bilayer. The hydrophilic layer of said liposome encapsulates model drug such as, but not limited to, Doxorubicin and Calcein, and the hydrophobic layer of said liposome encapsulates photosensitive substance to trigger the lipid bilayer instable and enhance the release of the drug of said liposome.

The present invention relates to a lipid bilayer of a liposome combination encapsulates hydrophobic photosensitive substances, said hydrophobic photosensitive substance is one of the Porphyrin group, but not limited to any photosensitive substance. One of the embodiments of the present invention is using Hematoporphyrin (Hp) and Protoporphyrin IX (PpIX) of the Protoporphyrin (Pp) group; and the hydrophilic layer of said liposome encapsulates hydrophilic drug such as, but not limited to, Doxorubicin and Calcein.

Another purpose of the present invention is related to activate the photosensitive substance of the lipid bilayer of said liposome by using a light with special wavelength to produce singlet oxygen and free radical and oxidized the lipid, and then said liposome releases out the anticancer drug. The singlet oxygen and the free radical will attack the cancer cells at the same time as a result of combining the photodynamic- and chemo-effects.

Another purpose of the present invention is related to a method for using said liposome combination. The present invention is using a light to enhance the release of the drug from said liposome. The light of the present invention is the light with longer wavelength such as, but not limited to, 600 nm-670 nm. One of the embodiments of the present invention is using red light to be the light source. Another embodiment of the present invention is using red light LED to be the light source.

### EXAMPLE 1

### Liposome Combination Preparation

Phospholipids, cholesterol, and PEG-DSPE dissolved in organic solvent were mixed with 600 μl of 0.5 mg/ml hematoporphyrin ethanol solution and rotary vacuum evaporator was used to remove the solvent. After removing the organic solvents, the lipid film was formed, and pre-heated doxorubicin aqueous solution (65° C.) was added for hydration. Repeated freeze-thaw process was performed and the size of the liposomes was controlled using a ultrasonic probe (20 mins, 50 w). Uncapsulated liposomes and drugs were removed using a Sephadex G-50 Column. The finial solution was suspended in 0.9% (w/v) NaCl and stored at 4° C.

### EXAMPLE 2

### Liposome Combination Preparation

The above prepared liposome combination was divided into 6 groups (each of 200 μ), separately sealed into 1.5 ml centrifuge tube, filled with argon and stored in dark under 4° C. On 0 day, 1^{st} day, 3^{rd} day, 7^{th} day, 15^{th} day and 30^{th} day, one group of the sample was removed for drug leakage (%) analysis the and particle size change.

Leakage (%)=ELp/CLp×100%

ELp=The drug content of liposome in experimental group

CLp=The drug content of liposome in controlled group (0 day)

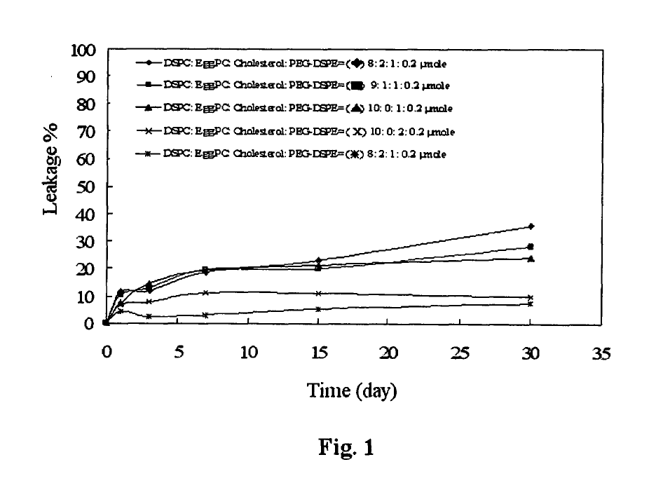

FIG. 1 shows the stability of liposomes after 30 days of storage. The drug leakage of the liposome combinations (Ch1, Ch2 and Ch3) of the present invention were within 20%. The drug leakage of Ch3 group was within 10%.

### EXAMPLE 3

### Liposome Combination Photo Triggered-Release

(1) Hematoporphyrin as the photo-triggering photosensitizer

The prepared Hp liposome combination were placed into a dialysis bag after illumined for 10, 20, and 30 J/cm² using a red light-emitting diode array device (LED). The dialysis solution was PBS, pH 7.4, and the dialysis was controlled at 37° C. One ml dialysis solution was taken from the dialysis bag and replaced with fresh buffer solution at 1, 2, 4, 6, 8, 12, 24, 36, 48, 72 hr after the triggered release. The collected solution containing released doxorubicin was quantitated by spectrofluorometer.

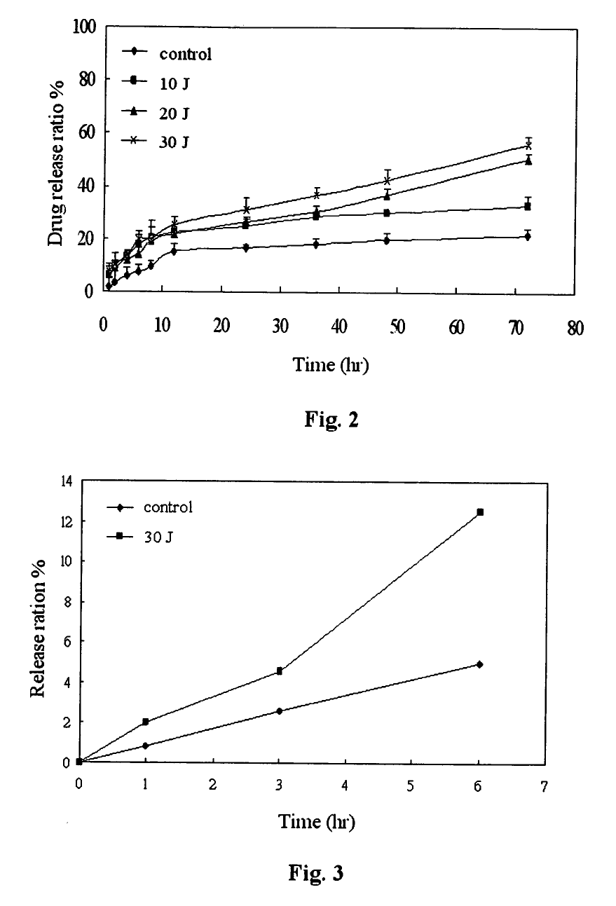

The result shows in FIG. 2 revealed that after illumination, the release of doxorubicin from the liposome combination with hematoporphyrin increased significantly. The release curve of liposome after illumination could be divided into 2 stages, and the turning point of the rate occurred about the 12^{th} hours. The drug release of liposome after 10, 20, 30 J/cm² illumination was 33%, 50%, and 56% in 72 hours. After 72 hours, the group of 30 J/cm² irradiation increase 36% compared to the control group. Hence, phototriggered reaction could enhance the release of doxorubicin from the liposome, and the Doxorubicin release was directly related to the intensity of illumination.

(2) Protoporphyrin IX (PpIX) as the photo-triggering photosensitizer.

Phospholipids, cholesterol, and PEG-DSPE dissolved in organic solvent were mixed with PpIX ethanol solution and a rotary vacuum evaporator was used to remove the solvent. After removing the organic solvents, the lipid film was formed, and pre-heated doxorubicin aqueous solution (65° C.) was added for hydration. Repeated freeze-thaw process was performed and the size of the liposomes was controlled by ultrasonic (20 mins, 50 w). Uncapsulated drugs were removed using a Sephedex G-50 Column. The finial solution was suspended in 0.9% (w/v) NaCl and stored at 4° C.

The prepared PpIX liposome combination were placed into a dialysis bag after illumined for 10, 20, and 30 J/cm² using a red light-emitting diode array device (LED). The dialysis solution was PBS, pH 7.4, and the dialysis was conducted at 37° C. 1 ml dialysis solution was taken from the dialysis bag and replaced with fresh buffer solution at 1, 2, 4, 6, 8, 12, 24, 36, 48, and 72 hr after the triggered release. The collected solution containing released doxorubicin was quantitatively analyzed by using a spectrofluorometer.

FIG. 3 was showed the release profile of doxorubicin from the liposomes with and without 30 J of red-light illumination (635 nm). The rhombus curve represents the control group, and the square curve represents the experiment group. The release of doxorubicin increased significantly after light illumination.

### EXAMPLE 4

### Permeability Change of Liposome After Light Excitation

Liposome was prepared without any drug. The formulation of liposome combination is DSPC: Cholesterol: PEG-DSPE=10:3:0.2 μM, and the preparation and storage condition is the same as previously mentioned. The liposome sample (1 ml) was mixed 1 ml of 0.25 mg/ml fluorescent substance (Doxorubicin or Calcein). The experimental group was treated with light irradiation (30 J/cm² red LED), and the controlled group was shielded in dark. If light exposure would affect the permeability of the liposome, the fluorescent substance would enter the liposome. The Sephedex G-50 column was used to separate the liposome and the untrapped fluorescent substance. The untrapped and trapped fluorescent substance was analyzed by fluorospectrometer for mass balance. The permeability change was presented by quantifying the trapped fluorescent substance and corrected by the quantity of phospholipids.

Preparation of a liposome combination with the photosensitizer (Hp). The lipid composition is DSPC: Cholesterol: PEG-DSPE=10:3:0.2 μM and encapsulated 0.3 mg Hp, liposome preparation and storage condition was the same as previously mentioned except that Hp was encapsulated into the liposome. The permeability test was followed the previous paragraph.

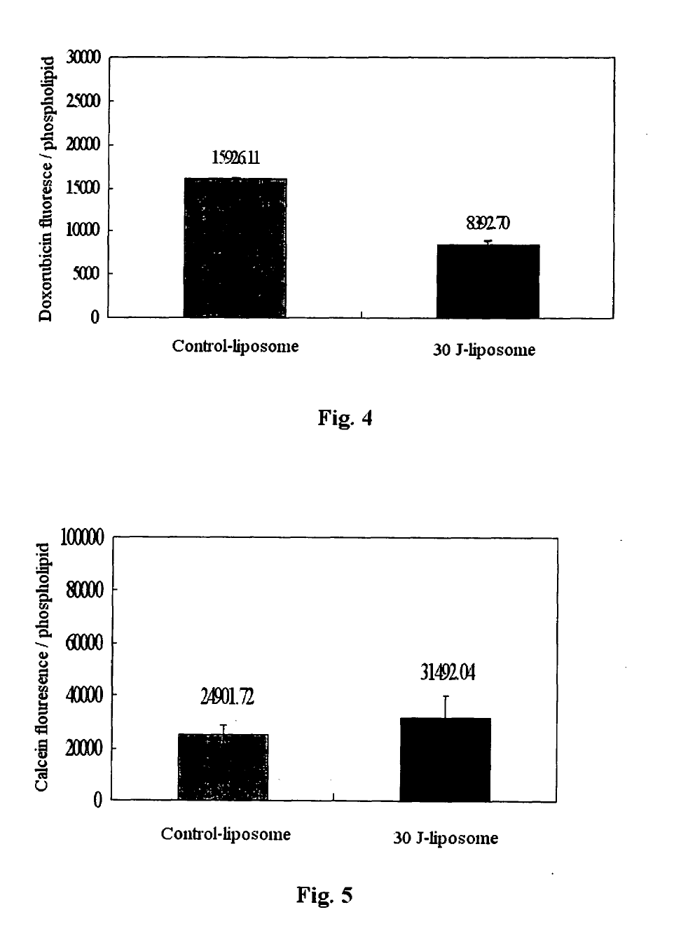

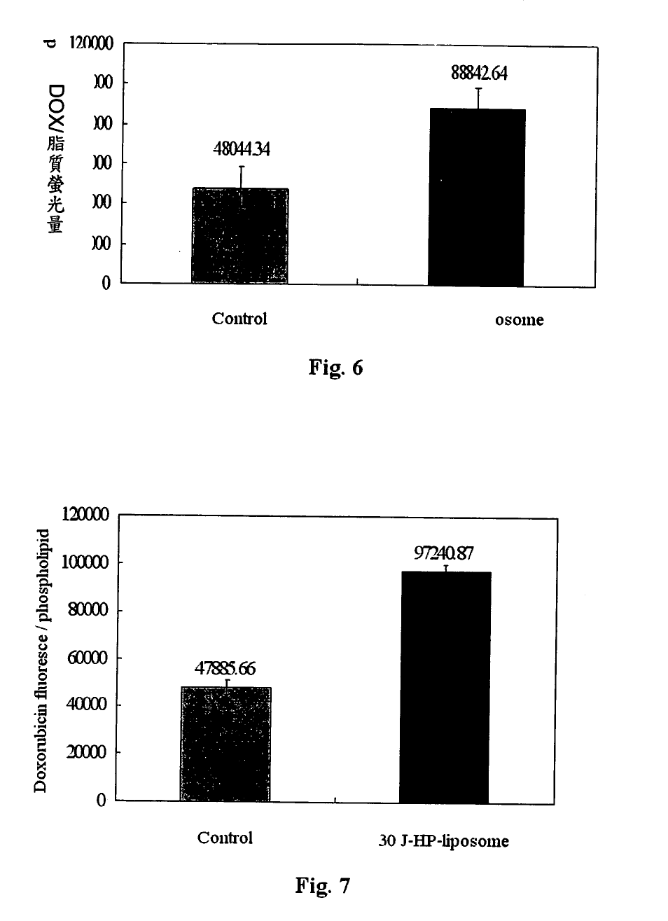

Permeability results of the photosensitizer-free liposome are shown in FIGS. 4 and 5, and the results of the Hp-liposome are shown in FIGS. 6 and 7. The amount of fluorescent substance in liposome was not increase in the same photosensitizer-free group, but the Hp-liposome group has opposite result. These results proved that Hp-liposome was treated with red right (30 J/cm²), the content of Doxorubicin or Calcein in liposome was increased significantly (almost doubled) compared to the corresponding controlled group.

### EXAMPLE 5

### Cell Culture and MTT Assay

(1) MTT Assay-1

CL 1-0 cells (approx. 5000 cells) were seeded in each well of a 96-well plate and incubated in medium. After 24 hr of incubation, 2 μg/ml Hp (as free or liposome) was added into the well. After 2 hr, the cells were illuminated for 2, 4, 6, or 8 J/cm² (635 nm red light), and MTT assay was performed 24 hr later.

(2) MTT Assay-2

A431 or CL1-5 cells (approx. 5000 cells) were seeded in each well of a 96-well plate and incubated in medium. After 24 hr of incubation, Doxorubicin (FD), Liposomal-Doxorubicin (LD) and Liposomal-Doxorubicin-Hematoporphyrin (LDH) was added into each well and drug concentration was set at 0.5 μg/ml Doxorubicin and 0.3 μg/ml Hp, After 2 hr, the cells of experiment group were illuminated for 10, 20, or 30 J/cm², and MTT assay was performed 72 hr later.

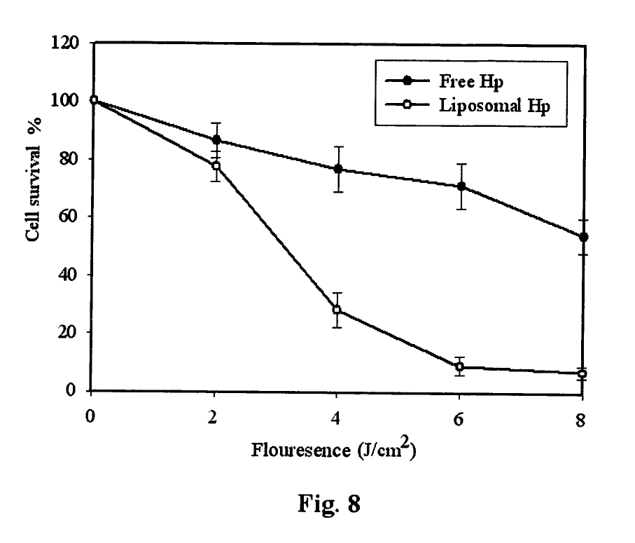

In the result of the cell experiment (1) (FIG. 8), compared the toxicity of free Hp with liposomal Hp with lung gland cancer cell (CL1-0) after light treatment, it can be find that the cytotoxicity effect of liposomal Hp to cancer cell is higher than that of free Hp significantly after light treatment. The result proves that the photodynamics therapy has poison killing effect to cancer cell, and the liposomal Hp can enhance the effect of photodynamics therapy to cancer cell.

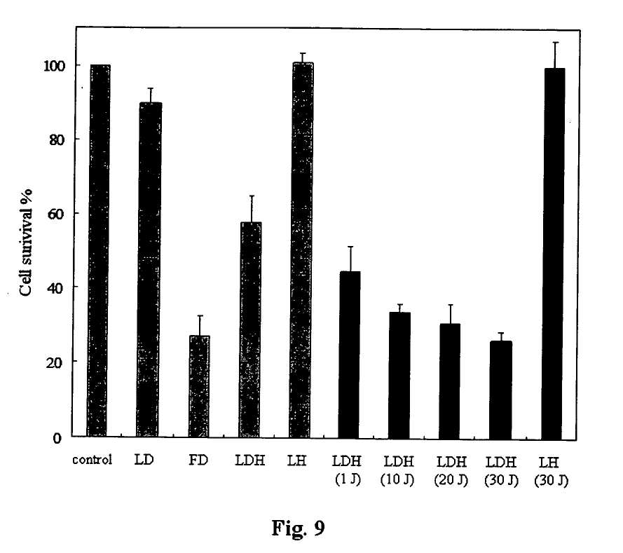

In the result of the cell A431 experiment (FIG. 9), it can be find that the toxicity of the cell with LD is lower than FD that was at the same concentration, It almost can be said that LD is not toxicity, and FD has toxicity to cell A431 significantly, the activity of mitochondia dehydrogenase of FD is about 30% activity of control group. The toxicity of liposomal-Doxorubicin-Hp (LDH) lies between LD and FD, the activity of mitochondia dehydrogenase of LDH is about 60% activity of control group. The toxicity of LDH has increasing after 1, 10, 20, and 30 J/cm² red light treating, and it seems in direct proportion to the intensity of illumination, especially in group 30 J/cm², the toxicity of said group is equivalent and/or stronger than FD, the activity of mitochondia dehydrogenase of LDH is about 25% f activity of control group. It is proved that the toxicity of LDH was increased because of the result of a large number released of anticancer drug from liposome or PDT effect after illumination. In cytotoxicity experiment (1), it is proved that liposome can enhance the poison killing effect of photodynamics to the cancer cell, thereof, it can reaches said effect by enhancing the HP doses, if it combines photodynamics and chemistry therapy for cancer cell.

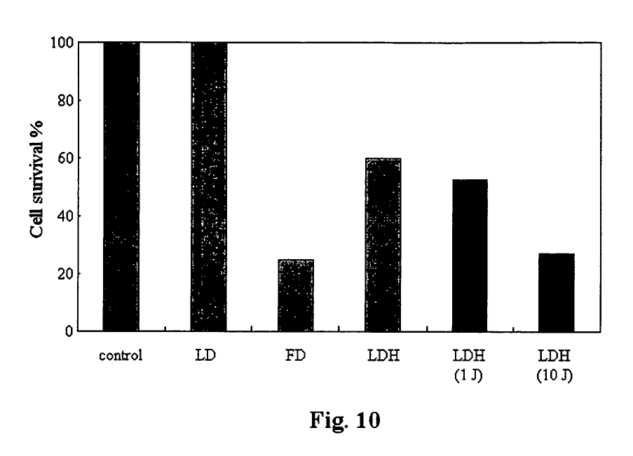

In the toxicity test of the cell CL1-0 (FIG. 10), the result is similar to the toxicity of the A431 cell. The cytotoxicity of LD is lower than FD does at the same concentration, LD has not toxicity almost at concentration of 0.5 μg/ml Doxorubicin, and FD has toxicity to cell CL1-0 significantly. The LDH toxicity of cell has increased after 1 and 10 J/cm² red light treating, and the result proves LDH liposome is useful in any kinds of cancer cell.

While the present invention has been described above in terms of specific embodiments, it is to be understood that the invention is not limited to these disclosed embodiments. Many modifications and other embodiments of the invention will come to mind of those skilled in the art to which this invention pertains; they are intended to be and are covered by both this disclosure and the appended claims. It is intended that the scope of the invention should be determined by proper interpretation and construction of the appended claims and their legal equivalents, as understood by those skilled in the art relying upon the disclosure in this specification and the attached drawings

1. A liposome combination comprising:
(a) a liposome
(b) a hydrophilic drug that is encapsulated in a hydrophilic layer of a liposome; and
(c) a hydrophobic drug that is encapsulated in a lipid bilayer of said liposome;
wherein said hydrophobic drug is a photosensitive substance selected from porphyrins which can excite said hydrophilic drug release by photodynamic action when said liposome is illuminated from a light source, and
wherein the liposome comprises phospholipids with saturated fatty acid.

2. (canceled)

3. The combination of claim 1, wherein said photosensitive substance is hematoporphyrin.

4. The combination of claim 1, wherein said photosensitive substance is protoporphyrin.

5. The combination of claim 1, wherein said light source is a red light.

6. The combination of claim 4, wherein the wavelength of said red light is 600 to 670 mm.

7. The combination of claim 6, wherein the red light source is 635 mm.

8. The combination of claim 4, wherein said red light source is a red light-emitting diode.

9. A method of using a combination of liposome as a drug releasing control system, comprising:
(a) exciting a photosensitive substance encapsulated in the hydrophilic layer in the lipid bilayer of said liposome by a light source;
(b) producing free radicals and singlet oxygen from said excited photosensitive substance;
(c) reducing the stability of said liposome by said free radicals and singlet oxygen; and
(d) releasing a drug that is encapsulated in the hydrophilic layer of said liposome,
wherein said photosensitive substance is selected from porphyrins and,
wherein the liposome comprises phospholipids with saturated fatty acid.

10. The method of claim 9, wherein said light source is a red light source.

11. The method of claim 10, wherein the wavelength of said red light source is 600 to 670 mm.

12. The method of claim 10, wherein said red light source is a red light-emitting diode.

13. (canceled)

14. The method of claim 9, wherein said photosensitive substance is hematoporphyrin.

15. The method of claim 9, wherein said photosensitive substance is protoporphyrin.

16. A method of using a liposome formulation combination in a photodynamic therapy and chemotherapy, comprising:
(a) using a light to excited a liposome exciting a photosensitive substance encapsulated in the hydrophilic layer in the lipid bilayer of said liposome by a light source;
(b) producing free radicals and singlet oxygen from said excited photosensitive substance;
(c) reducing the stability of said liposome and causing cytotoxicity by said free radicals and singlet oxygen; and
(d) releasing a chemotherapy drug that is encapsulated in the hydrophilic layer of said liposome;
wherein said photosensitive substance is selected from porphyrins and,
wherein the liposome comprises phospholipids with saturated fatty acid.

17. The combination of claim 1, wherein said phospholipids with saturated fatty acid is 1,2-disteroyl-sn-glycero-3-phosphocholine.

18. The method of claim 16, wherein said phospholipids with saturated fatty acid is 1,2-disteroyl-sn-glycero-3-phosphocholine.

19. The method of claim 9, wherein said phospholipids with saturated fatty acid is 1,2-disteroyl-sn-glycero-3-phosphocholine.
